(19) United States
(12) Patent Application Publication
(10) Pub. No.: US 2020/0001084 A1
(43) Pub. Date: Jan. 2, 2020

(54) UNIVERSAL MODULARIZED PORTABLE THERAPEUTIC LIMB AND BODY REST PAIN MANAGEMENT APPARATUS

(71) Applicant: Karen Robinson, Baldwin, NY (US)

(72) Inventor: Donald Ira Harrison, JR., Baldwin, NY (US)

(21) Appl. No.: 16/450,914

(22) Filed: Jun. 24, 2019

Related U.S. Application Data
(60) Provisional application No. 62/689,827, filed on Jun. 25, 2018.

(51) Int. Cl. A61N 1/36; A61N 1/04

(52) U.S. Cl. CPC A61N 1/36021; A61N 1/0456; A61N 1/36034; A61H 2201/0107; A61F 7/02; A61H 2201/02; A61H 2201/5046; A61H 2201/165; A61H 2201/0157

(57) ABSTRACT

A universal modularized portable therapeutic limb and body rest pain management device is disclosed. The portable therapeutic pain management device is coupled with a biostable spring slap mechanism which allows the device to be adjusted to enclose a user's limb or body part at a convenient location.

### PRIORITY CLAIM

This patent application is a Non-Provisional patent application and claims priority under 35 U.S.C. § 119(e) to the following applications: U.S. Provisional Patent Application Ser. No. 62/689,827, titled “Universal and Modularized Portable Therapeutic Limb/Body Rest” filed Jun. 25, 2018. The entire disclosure of the afore-mentioned patent application is incorporated by reference as if fully stated herein.

### FIELD OF THE INVENTION

The present invention, a universal modularized portable therapeutic limb and body rest pain management apparatus, is in the mechanical-electrical field and relates generally to a portable therapeutic apparatus for applying heat, cool, electrical stimulation and compression for pain-management and thermo-electro therapy of injuries to the limbs and body. The present invention is an alternative therapeutic apparatus to taking prescription drugs or undergoing invasive procedures.

This invention is a apparatus substance that comprises a primary pillow or pad filled with kinetic sand (or similar substance), the fabric of the primary pillow or pad being: Kevlar, nano-fibers, or similar fabric that is hypoallergenic and is capable of working in relation to the technologies incorporated along with silicone snap rubber which will be the outer layer of the apparatus. The invention provides electrical stimulation therapy, with an embedded central processing unit (chip) capable of recording, reporting, and managing data and the components of the apparatus. The invention also includes an artificial intelligence (“AI”) program installed in the apparatus that allows the recorded information to further assist physicians or physical therapists in treating patients. All aspects of the apparatus are made to be controlled by remote or an application on the end user's mobile apparatus.

### SUMMARY OF THE PRESENT INVENTION

The universal modularized portable therapeutic limb/body rest pain management apparatus is an all in one heating, cooling, transcutaneous electrical nerve stimulation technology (TENS), compression, vibration technology compacted in a therapeutic arm, leg or body rest that will not only be innovative towards the future of comfort of a user's injury but also will change the orthopedic/sports medicine industry as well as how physicians or therapists treat patients post-surgery.

This therapeutic pain management apparatus is designed around the user's injuries or pain, wherein the user may enclose the limb or body part within the apparatus and apply heat, cool, electrical stimulation, compression or massage modality to manage pain or treat an injury as may be prescribed by a physician or physical therapist. The apparatus will collect and store the user's application and use of the apparatus and the user's pain response to provide a report to the user's physician or therapist. Even though other methods currently exist for treating an individual with transcutaneous electrical stimulation, this apparatus would be an improvement on the art to augment or even replace current techniques.

An aspect of an embodiment of the present invention contemplates a first layer comprising kevlar, nano-fibers or nano-whiskers. These fabrics have been selected because of their adaptability as semiconductors. Another aspect of an embodiment of the present invention contemplates a second layer comprising kinetic sand enclosed inside of a leak proof designated fabric. Kinetic sand is also a semi-conductor which minimizes any melting or leakage when heated. Another embodiment is a third layer forming the base of the apparatus comprising silicone snap rubber or polypropylene, which is also beneficial because of their unique adaptability to perform as semiconductors. A further embodiment contemplates flaps on both the left and right sides of the apparatus which will either snap over or fold over the limb or body part enclosing it with a locking mechanism.

An aspect of an embodiment of the present invention contemplates an adjustable tripod stand wherein the tripod adjustable stand may include the following component parts or elements: a mounting board, locking buttons, rotating hinge and an adjustable length pole. In an aspect, the tripod adjustable stand may be adjusted to suit the comfort level of the user.

In another aspect of an embodiment of the present invention, a microchip with an artificial intelligence (AI) program is embedded within the layer of the apparatus, wherein the microchip is configured collect usage data to analyze stressed and/or affected limb or body parts to communicate the information to a medical professional or physical therapist (PT) in order for the medical professional or therapist to adjust their professional recommendations towards the application of the treatment modalities to deliver the appropriate heat, cold, -electro stimulation, compression or massage more effectively. The PT or medical professional can utilize the analysis to prescribe electrical shock treatment to help with nerve damage, control the temperature of this invention from heat to cold without ice or heating pads, and/or give massages through gentle and slightly aggressive vibrations/compressions.

In another embodiment of the present invention, the embedded memory chip may further include a step to provide a daily progress report on the affected area and what the end user can do to adjust their comfort levels.

For a further and more fully detailed understanding of the present invention, various objects and advantages thereof, reference is made to the following detailed description and the accompanying drawings.

The foregoing and other objects and advantages will appear from the description to follow. In the description, references are made to the accompanying drawings, which forms a part hereof, and in which is shown by way of illustration specific embodiments in which the invention may be practiced. These embodiments will be described in sufficient detail to enable those skilled in the art to practice the invention, and it is to be understood that other embodiments may be utilized and that structural changes may be made without departing from the scope of the invention. In the accompanying drawings, like reference characters designate the same or similar parts throughout the several views.

The following detailed description is, therefore, not to be taken in a limiting sense, and the scope of the present invention is best defined by the appended claims.

### BRIEF DESCRIPTION OF THE DRAWINGS

Further objectives and advantages of the present invention may be derived by referring to the detailed description and claims when considered in connection with the Figures, wherein like reference numbers refer to similar items throughout the Figures.

FIG. 1 illustrates the complete apparatus in an open configuration displaying the therapeutic cells and sensors for conduction of the heat, cool, electrical-stimulation and compression attached to the tripod stand.

FIG. 2 illustrates a blow-apart diagram of the portable apparatus according to an aspect of an embodiment of the present invention, showing four components with their configurations and functions in segmented form: nano-whisker fabric 1 (top portion), kinetic sand 2 (second portion), canvas or similar fabric 3 (third portion), and silicone snap rubber 4 or similar substance for example, polypropylene snap rubber (fourth portion) forming the external portion of the apparatus.

FIG. 3A illustrates how the sides, or flaps 5 (smaller rectangles), fold up and around the leg or body part, thus cuffing it. That is the top portion and fourth portion will fold up and around the limb or body part, all four portions moving as one-layer apparatus or apparatus according to an aspect of an embodiment of the present invention.

FIG. 3B illustrates how the sides, or flaps fold up without the enclosed limb or body part. It also illustrates the adjustable tripod stand according to an aspect of an embodiment of the present invention.

FIG. 3C illustrates an enclosed limb or body part with the flaps folded and with the adjustable tripod stand according to an aspect of an embodiment of the present invention.

FIG. 4 illustrates the curvature of one of the flaps attached to the larger segment into which the limb or body part is placed within the apparatus according to an aspect of an embodiment of the present invention.

FIG. 5A illustrates an aerial view of how a limb or body part is first set on top of the center piece, with the flaps folded around it and fastened. The flaps close and seal the limb or body part within the apparatus according to aspects of embodiments of the present invention.

FIG. 5B illustrates a segmented view of the apparatus in an enclosed position with the flaps folded and fastened with the tripod stand according to aspects of embodiments of the present invention.

FIG. 5C illustrates a side profile view of an enclosed limb with the flaps folded and fastened and with the adjustable tripod stand according to aspects of embodiments of the present invention.

FIG. 6A [-6C] illustrates an aerial view of how a limb or body part is first set on top of the center piece, with the flaps folded around it and fastened and the adjustable tripod stand according to an aspect of an embodiment of the invention.

FIG. 6B illustrates a segmented view of the apparatus without an enclosed limb or body part illustrating the flaps folded and fastened according to aspects of embodiments of the present invention.

FIG. 6C illustrates an enclosed limb or body part elevated with the adjustable tripod stand according to aspects of embodiments of the present invention.

FIG. 7 illustrates individual therapeutic cells & sensors collated; which facilitate the various modalities as well as sensors analyzing the affected areas. Affected area will be analyzed by sensors as users directs touchscreen controller to apply the different treatment modalities to the affected area. The AI component can focus on the affected area and give algorithmic feedback on better assisting the injury.

FIG. 8 illustrates a process flow showing operation of a portable apparatus or apparatus according to an aspect of an embodiment of the present invention.

### DETAILED DESCRIPTION OF THE PREFERRED EMBODIMENT

The following discussion describes in detail one embodiment of the system and method of the invention (and several variations of that embodiment). However, this discussion should not be construed as limiting the invention to those particular embodiments as practitioners skilled in the art will recognize numerous other embodiments as well. For definition of the complete scope of the invention, the reader is directed to appended claims. Turning now descriptively to the drawings, in which similar reference characters denote similar elements throughout the several views.

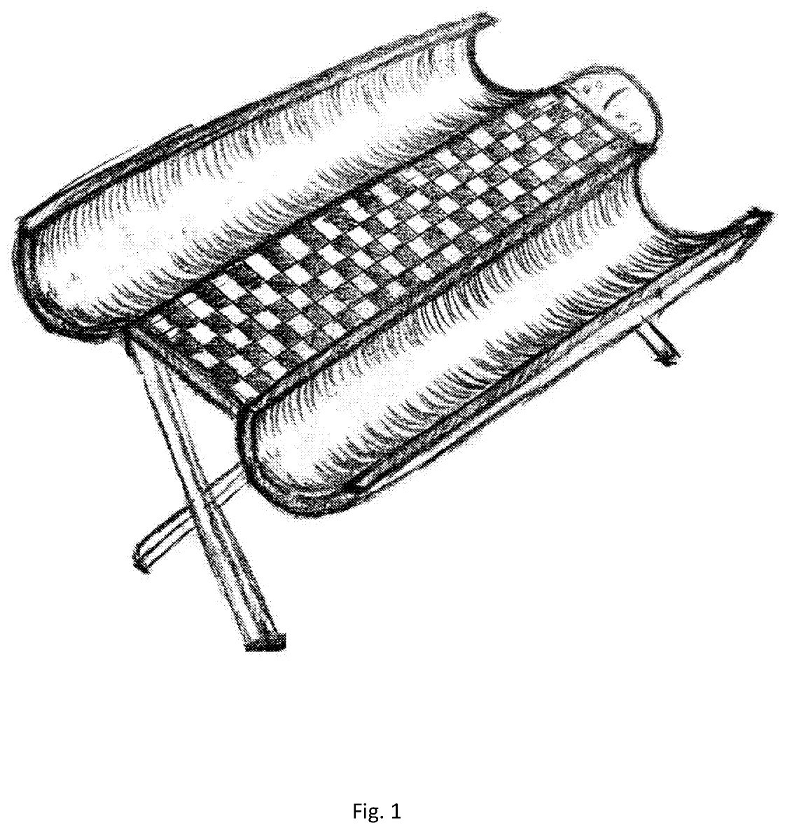

Referring now to FIG. 1, which contains an illustration of the complete apparatus in an open configuration displaying the therapeutic cells and sensors for conduction of the heat, cool, electrical-stimulation and compression attached to the tripod stand.

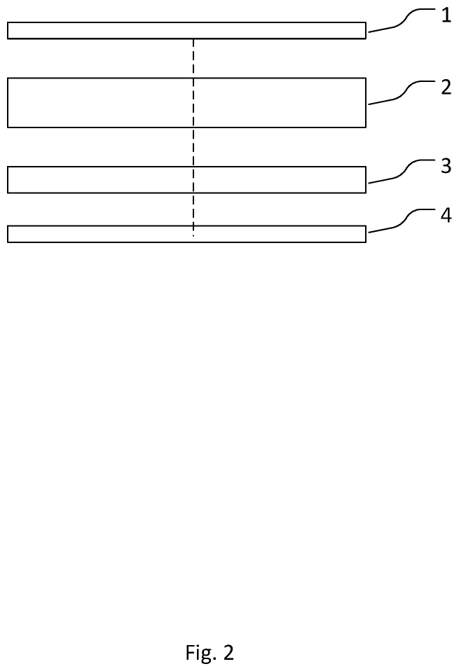

Referring now to FIG. 2, which contains the aerial view of the compilation materials: the first portion and inner layer of the apparatus being the kevlar, nano-whiskers, nano-fibres or similar eco-friendly hypoallergenic material, second portion comprising kinetic sand or similar memory foam cushion enclosed in a leak-proof bag, third portion comprising canvas material to add support and fourth portion forming the exterior portion of the apparatus is the silicone snap rubber, polypropylene snap rubber or similar material forming the flaps of the apparatus allowing for the apparatus to enclose the limb.

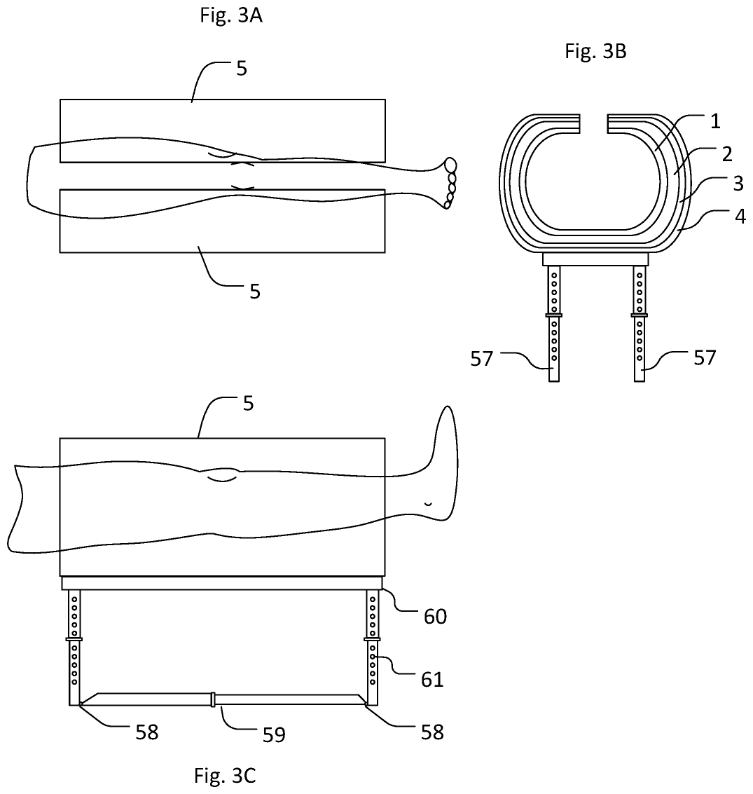

Referring now to FIG. 3A which contains an aerial view of leg in apparatus, opened with flaps 5 released. FIG. 3B shows the apparatus enclosed without leg or body part in the apparatus. FIG. 3B also shows the layers composed of nanowhiskers 1, kinectic sand 2, canvas support layer 3, silicone snap rubber 4 mounted on the adjustable poles 57. FIG. 3C illustrates a leg enclosed by the flaps 5 of the apparatus, the mounting board 60, with locking buttons 61, rotating hinge 58, and adjustable length pole 59.

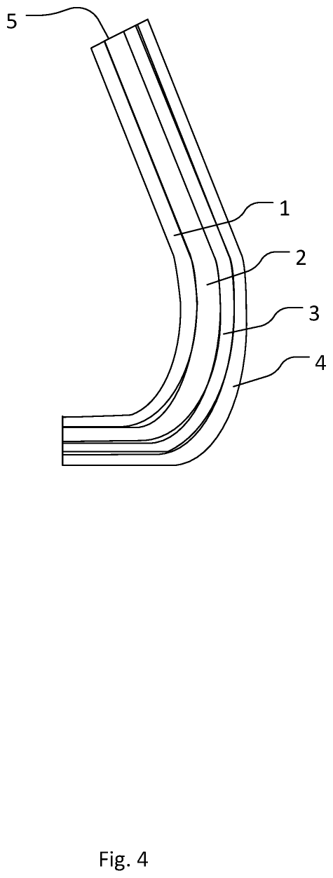

Referring now to FIG. 4 which illustrates a zoomed in compilation of layered materials starting with the inner layer: nano-whiskers or hypoallergenic or similar fabric 1, kinetic sand, memory foam or similar cushion 2, canvas or similar material to add support 3, silicone snap rubber or similar material 4 which serves as the flaps allowing the apparatus to snap to enclose the limb or body part.

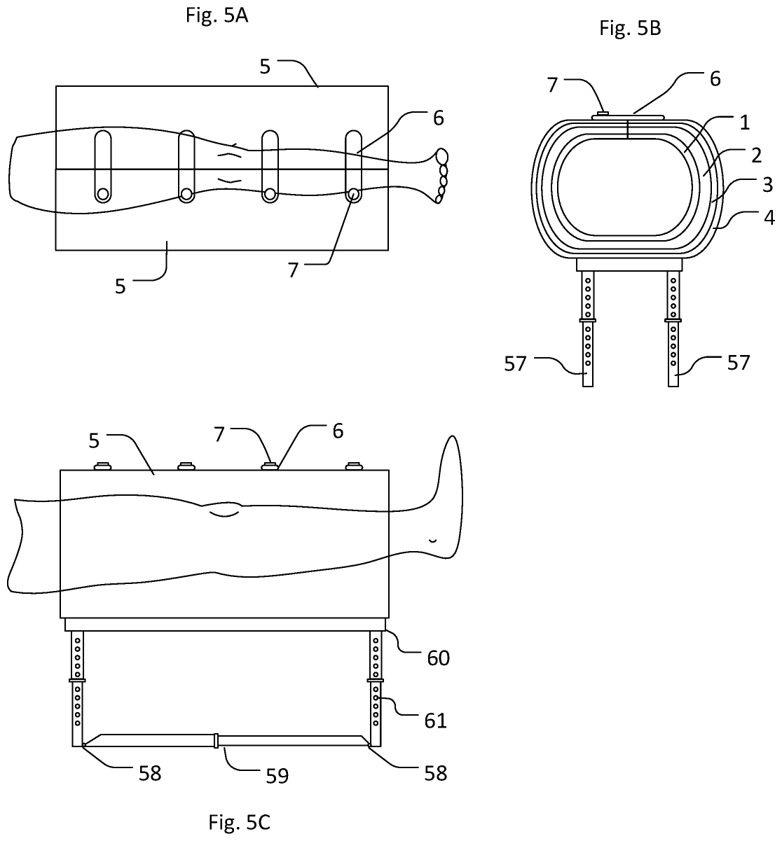

Referring now to FIG. 5 which illustrates an aerial (5A) and side views (5C) of a limb in the apparatus locked in place by the flaps 5 and ready for usage. 5B illustrates a side view of the apparatus illustrating the inner layer 1, kinetic sand 2, canvas 3, and silicone snap rubber 4 locked without leg/limb enclosed in the apparatus.

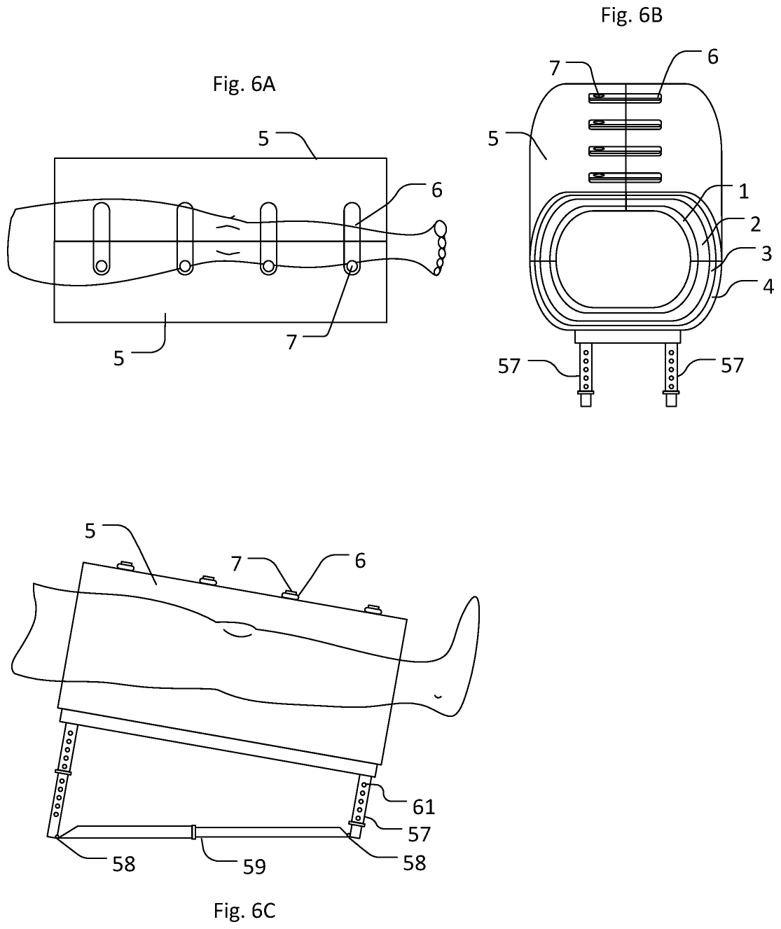

Referring now to FIG. 6 which illustrates aerial (6A) and side views (6B) of a limb enclosed in the apparatus being used in an elevated mode (6C) using the adjustable tripod stand.

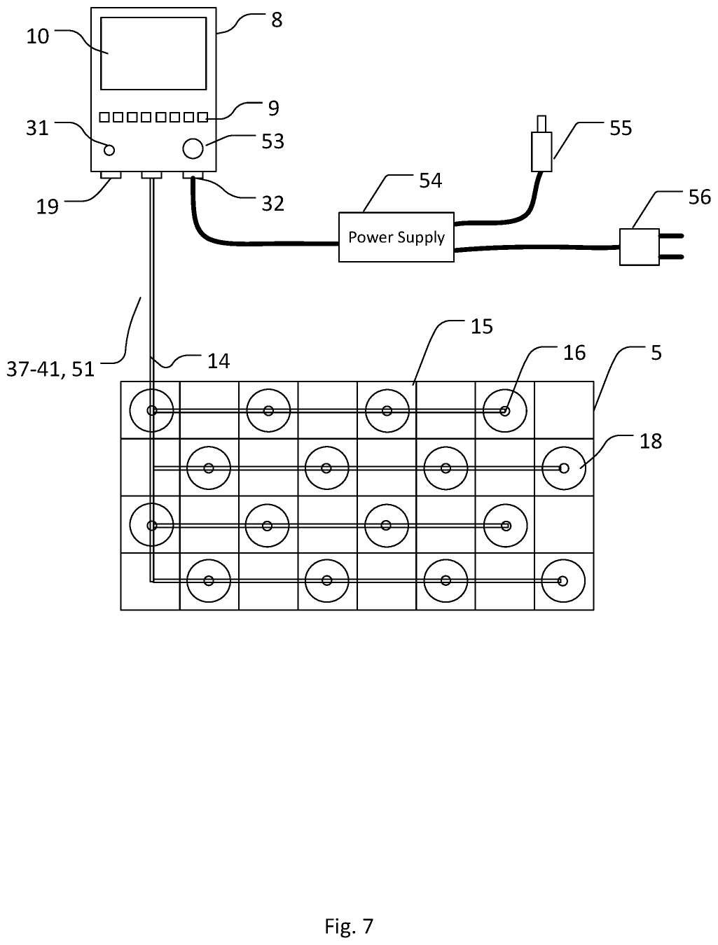

Referring now to FIG. 7 shows touch screen controller with attached power supply module. The controller is attached to the apparatus by an inflatable tube 14 to facilitate compression, electrical stimulation and the heating and cooling modalities to allow the end users to control the technologies. FIG. 7 also illustrates the schematic of a flap 5 (that may in one modality be exchangeable) indicating that it contains the therapeutic cells 15 and sensors 16 for conduction of the heat, cold, electrical stimulation and the compression therapeutic elements to provide electrical stimulation (TENS/EMS machine), heat/cool, compress, or vibrate, 18 as indicated by the succession of icons displayed on the controller, each or a combination of which can be mapped on to any or a plurality of squares in the checkerboard rectangle. The affected area will be analyzed by sensors as users directs touchscreen remote to where the affected area is, and the AI component can focus on the area and provide algorithmic feedback on better assisting the injury.

FIG. 8 illustrates the controller module 8 which circuits a charger port 32 which connects to the power supply 54. The connector interfaces with a mating connector on the power supply 52 cable. This charger port will feed the battery charger circuit 30. The battery charger circuit 30 is responsible for the safe charging of the battery pack 12. It contains various features such as reverse voltage, over voltage, and over charging protection. A multi-color LED 31 is connected to the battery charger circuit 30 to indicate the presence of power and the charging status. The battery charger circuit 30 connects to a rechargeable battery pack 12. This battery pack consists of multiple cells connected in parallel for increased power capacity. The rechargeable battery pack 12 connects to a battery protection circuit 29. This protection circuit 29 is intended to protect the battery pack 12 from extreme conditions such as rapid discharge and discharge below a designated safe voltage. The circuit will disconnect the battery 12 from the load when these extreme conditions occur.

The battery protection circuit 29 feeds a ON/OFF switch 28. This switch is accessible to the user and is used to power the controller module 8 ON and OFF. It disconnects all power from the rest of the circuitry in the controller module 8 when the switch is in the open position. The ON/OFF switch 28 feeds the power supply section 26. The power supply section 26 contains a few individual power supply circuits. These power supplies are used to power all the individual circuits in the controller module 8 with the proper regulated voltage. Item 27 is a power supply supervisor/sequencer. This circuit is responsible for the monitoring of all the individual power supply outputs. If any of the individual power supply outputs is out of regulation then it creates a fault status and sends it to the CPU 11. This circuit is also responsible for the sequencing of the individual power supplies during power up. This ensures the power supplies come on in the correct sequence. FIG. 8, also illustrates a keypad 9 that resides on the front surface of the controller module 8. This keypad 9 allows the user to interact with the controller module 8. These keys can be in various forms such as tactile dome push buttons or capacitive touch type. They can also be part of the touch display 10 and be virtual. The touch display 10 will provide the user with a graphical user interface (GUI). The display will be used to configure the controller module 8 operation. It will display multiple menus for the user to interact with. The touch display 10 can be designed using multiple capacitive touch technologies that currently exist. The Universal Serial Bus (USB) connector 19 can be one of multiple types of USB connectors that currently exist. It provides the user a means to communicate with the controller module 8 without using the wireless radio 13. This port can be connected to a PC to upload new software, download data, and download configurations. A USB drive can also be connected to it so data can be exported from the controller module 8.

The USB Connector 19 connects to a USB Interface 20. This interface 20 is a circuit that buffers the signals coming from the CPU 11 and converts them to the proper voltage levels for USB communications. It also provides transient voltage protection to prevent surges on the USB interface 19 from damaging the CPU 11. An antenna 25 is utilized by the wireless radio 13 for transmission of wireless signals. This antenna 25 can be designed to operate at various frequency bands depending on the type of wireless radio 13 connected to it. The antenna 25 will be contained within the enclosure of the controller module 8. The wireless radio 13 is used to convert the digital communications from the CPU 11 to a frequency and power level that permits wireless transmission. This radio 13 could be one of various technologies such as WiFi or Bluetooth. The user can use wireless communications to control, configure, download, or upload data. It can be used to establish communications between a phone, tablet, or computer. The real time clock (RTC) 33 consists of a frequency source. This RTC 33 is required for the module 8 to keep accurate time for data logging of events. This RTC 33 is backed up with a separate battery 34. A separate battery is necessary in case the main battery becomes fully discharged. The Random-Access Memory (RAM) 24, is used by the CPU 11 for fast access of stored information. The software on the module runs out of the RAM 24. Temporary data is stored in the RAM 24 for fast access. This memory 24 is volatile and will be erased when the module 8 is powered off. This section 24 may consist of one or more memory integrated circuits (ICs). The Non-Volatile (NV) Memory 17, is used for long term data storage. This memory will be used to store the program that runs on the CPU. Upon power on, the software is loaded from the NV Memory 17 into the RAM 24. Event logging, configuration data, and preferences are all stored on the NV Memory 17.

The Analog to Digital Converter (ADC) 21 is used to convert analog signals coming from the various temperature and pressure sensors. These signals can be very low level when they come from the sensors. These low-level signals come in through the multi-pin connectors 37 and 38. They are then boosted by the amplifiers 36 and 35. Once digitized, these signals are then passed on to the CPU 11 for processing. The CPU 11 controls the rate at which the ADC signals are sampled. The Digital to Analog Convertor (DAC) 22 is used to convert digital signals from the CPU 11 into analog signals. These analog signals are then buffered by the amplifier circuits 42-46. Amplifier 42 is used to drive multiple vibrators that are located within the cuff 5. These amplified signals travel through the multi pin connector 39. This connector then interfaces with the electrical leads 14. Amplifier 43 is used to interface with the heaters that are located within the cuff 5. These high-power outputs travel through the multi pin connector 40 and then on to the electrical leads 14. Amplifier number 44 is used to control the cooling apparatus located in the cuff 5. These high-power outputs travel through the multi-pin connector 40 and then go on to the electrical leads 14. Amplifier 45 is used to control the EMS circuitry 48. This amplifier 45 is used to control the amplitude of the EMS pulses. Amplifier 46 is used to control the TENS circuitry 47. This signal coming out of amplifier 46 is used to control the amplitude or strength of the TENS pulses.

The Central Processing Unit (CPU) 11 is used to run the module software. It also interfaces with all the major sections of the control module 8. It loads the software when it is powered on and transfers it to the NV memory 17. The CPU 11 also communicates with the A to D convertor 21 and the DAC 22. The CPU 11 has various digital outputs 23 that are used to control various hardware such as the piezo 53, air compressor 50, EMS pulses, and TENS pulses. Some of these outputs are pulse width modulated (PWM). The CPU 11 communicates with external apparatus[s] through the USB 20 and wireless interfaces 13. It transmits data to the display 10 and receives touch information from it. It accepts inputs from the keypad 9.

The TENS amplifier 47 receives digital pulses from the CPU 11. It boosts these signals and drives the electrodes that are in the cuff 5. The amplitude of these signals is controlled by the analog signal coming from amplifier 46. The TENS outputs travel through the multi-pin connector 41. They then pass on to the electrical leads 14.

The EMS amplifier 48 receives digital pulses from the CPU 11 and boosts them so they can drive the electrodes located in the cuff 5. The amplitude of these pulses is controlled by the analog signals coming from amplifier 45. These signals then travel through the multi-pin connector 41 and connect to the electrical leads 14.

The air compressor 50 is used to compress air in the hollow cavities of the cuff 5. This compressor 50 is controlled by the compressor driver 49. The compressor driver circuitry 49 is driven by the CPU 11. The output of the air compressor 50 is connected to a hose connector 51.

The hose connector 51 is located on the wall of the module enclosure 8. The piezo driver 52 receives digital signals from the CPU 11 and amplifies them. The output is then connected to a piezo transducer 53. This piezo transducer 53 is located on the wall of the enclosure 8 so it can be heard by the user. It is used to provide audible feedback to the user.

While the principles of the disclosure have been described herein, it is to be understood by those skilled in the art that this description is made only by way of example and not as a limitation as to the scope of the disclosure. Other embodiments are contemplated within the scope of the present disclosure in addition to the exemplary embodiments shown and described herein. Modifications and substitutions by one of ordinary skill in the art are considered to be within the scope of the present disclosure.

What is claimed is:

1. A portable heat, cold, thermo-electro stimulation compression apparatus for pain management, the apparatus comprising:
a layer including first, second, third and fourth portions having respective edges that are connected to each other while being spaced apart from each other by a separator;
a therapeutic apparatus configured to be positioned between the layer and an extremity or body part of a user when the extremity or body part is positioned within an opening defined by the layer; and
a biostable spring slap flap connected to the fourth portion of the layer; and
a locking mechanism attached to the biostable spring slap flap; and
a microchip disposed within the apparatus; and
a touch screen controller; and
a rechargeable battery pack; and
an adjustable tripod stand;

2. The portable thermo-electro stimulation compression apparatus recited in claim 1, wherein the first portion is comprising kevlar, nanowhiskers, or nanoparticles wherein the Kevlar, nanowhiskers, or nanoparticles are eco-friendly and resistant to hot or cold temperature; the second portion comprising kinectic sand enclosed in a leak proof fabric, the third portion is comprising canvas for support, and the fourth portion comprising silicone or polypropylene snap rubber.

3. The apparatus as recited in claim 1, wherein the biostable spring slap is a tightening mechanism comprised of silicone or polypropylene snap rubber;

4. The apparatus as recited in claim 1, further comprising vibrators, cooling and heating coils, diathermy, piezoelectric apparatus or a TENS unit disposed between the silicone or polypropylene snap rubber and the second portion comprising the kinetic sand.

5. The apparatus as recited in claim 1, wherein the layer has two free ends, and the biostable spring slap tightening mechanism releasably connects the two free ends together secured by a locking mechanism comprising velcro.

6. The apparatus as recited in claim 1, wherein the tripod adjustable stand is juxtaposed to the fourth portion.

7. The apparatus as recited in claim 7, wherein the tripod adjustable stand comprises a mounting board, locking buttons, rotating hinge and an adjustable length pole.

8. The apparatus of claim 2 wherein the touchscreen controller comprises a navigational pad enabling the user to navigate through different operation and capabilities of the apparatus.

9. A computer program product comprising a computer readable medium having user logics stored therein for causing a processor to enable the display of collected data by an apparatus for displaying data, the user logic comprising computer readable code for:
receiving usage information from a user; and
receiving and processing stored data; and
provide algorithmic feedback to improve usage of the apparatus.

10. A computer program product of claim 9 further comprising of computer readable code for processing received usage information and determining whether usage modalities may be adjusted.

11. A computer-implemented method for operation of an apparatus for collating user's usage of the apparatus, comprising executing on a processor the steps of:
receiving usage information from a user; and
receiving and processing stored data; and
provide algorithmic feedback to improve usage of the apparatus.

12. A computer-implemented method of claim 11 further comprising a step of processing the stored data and determining whether user's treatment modalities may be adjusted.

13. A computer-implemented method of claim 12 wherein the step of determining whether user's treatment modalities may be adjusted further comprises of a step of comparing the stored usage data with previously stored information.
